(19) United States
(12) Patent Application Publication
(10) Pub. No.: US 2026/0151376 A1
(43) Pub. Date: Jun. 4, 2026

(54) COMBINATION THERAPY FOR THE TREATMENT OF CANCER

(71) Applicants: INSTITUT NATIONAL DE LA SANTÉ ET DE LA RECHERCHE MÉDICALE, Paris (FR); UNIVERSITE COTE D'AZUR, Nice (FR); BIPER THERAPEUTICS, Strasbourg (FR); CENTRE NATIONAL DE LA RECHERCHE SCIENTIFIQUE, Paris (FR)

(72) Inventors: Cyril RONCO, Nice Cedex 2 (FR); Rachid BENHIDA, Paris (FR); Stéphane ROCCHI, Paris (FR); Mehdi CHELBI, Strasbourg (FR)

(21) Appl. No.: 19/121,677

(22) PCT Filed: Oct. 19, 2023
(86) PCT No.: PCT/EP2023/079205

§ 371 Date: Apr. 16, 2025

(30) Foreign Application Priority Data
Oct. 20, 2022 (EP) 22306589.7

(51) Int. Cl. A61K 31/426; A61K 39/00; A61K 39/395; A61P 35/00

(52) U.S. Cl. CPC A61K 31/426; A61K 39/3955; A61P 35/00; A61K 2039/505

(57) ABSTRACT

The present invention pertains to a combination of specific benzene sulfonamide thiazole compounds with an anticancer treatment for use in the treatment of cancer in a patient who has been identified as overexpressing GRP78. Using a colorectal cancer mice model (CT26 allograft model) and several cancer cell lines models in vitro, the present inventors have shown that a combined therapy using HA15, a benzene sulfonamide thiazole compound, and an anticancer treatment such as an immunotherapeutic agent, a chemotherapeutic agent or targeted therapies significantly improves the survival rate and strongly reduces tumor growth as compared to monotherapies comprising either a benzene sulfonamide thiazole compound or an anticancer agent alone.

### FIELD OF THE INVENTION

The present invention pertains to novel cancer therapies. The present invention particularly relates to a combination of an anticancer treatment with a benzene sulfonamide thiazole compound for use in the treatment of cancer in a patient who has been identified as overexpressing GRP78.

### BACKGROUND OF THE INVENTION

Cancer encompasses a large family of diseases characterized by uncontrolled cell growth and division. Together, the over 200 known forms of cancer inflict a terrible social burden in terms of loss of life, diminished quality of life, healthcare costs and reduced productivity. Significant improvements in the diagnosis, screening and treatment of cancer have been made in the last decade. Nevertheless, cancer is still a leading cause of death worldwide: it accounted for nearly 10 million deaths in 2020 (Ferlay et al. Global Cancer Observatory: Cancer Today. Lyon: International Agency for Research on Cancer; 2020).

Many types of cancer treatments have been developed and are available such as chemotherapy, radiotherapy and immunotherapy.

Chemotherapy is often part of the first-line anticancer regimen. Although showing good results in a large number of cancers, it is also accompanied by several adverse effects that can strongly impair patients' quality of life. A close management of the doses and regimens applied is essential for optimizing the chances of remission and maintaining the patient's well-being.

Targeted therapies developed for specifically targeting genes and molecules directly involved in carcinogenesis and tumor growth have also proven extremely useful. These therapies are a form of personalized medicine and contrary to traditional chemotherapy, they do not “simply” target any rapidly dividing cell. Targeted therapies are therefore supposed to be better tolerated by patients. Unfortunately, the identification of appropriate molecular targets is still in progress and the development of the corresponding drugs takes a lot of time.

Immunotherapy is now a recognized and well-established therapeutic alternative for treating cancer. It groups together several different therapies, all based on stimulating the immune system of the patient in order to recognize and attack his or her disease. Immunotherapy using immune checkpoint modulators has revolutionized the oncology field (Robert, C. Nat Commun 11, 3801, 2020). Studies at the origin of the concept of this type of therapy led to James P. Allison and Tasuku Honjo winning the Nobel Prize in Medicine in 2018. Immune checkpoints refer to a plethora of inhibitory pathways hardwired into the immune system that are crucial for maintaining self-tolerance and modulating the duration and amplitude of physiological immune responses in peripheral tissues in order to minimize collateral tissue damage resulting from immune responses. When the checkpoint and its ligand bind together, they send an “off” signal to the T cells, thereby inhibiting/strongly reducing immune responses. It is now clear that tumors use certain immune checkpoint pathways in order to escape immunity responses. Immune checkpoint inhibitors (ICI) work by blocking the checkpoint from binding with its ligand so as to prevent the “off” signal from being sent. These compounds have shown remarkable clinical efficacy and represent substantial hope for cure for some treatment-resistant tumors. Unfortunately, not all tumors respond to immune checkpoint inhibitors. In some cases, even after having turned off the inhibitory signal sent by immune checkpoints, the immune system is simply not capable of detecting and attacking the tumor. Some tumors are indeed non-immunogenic which means that they cannot be detected by immune cells. The immunogenicity of a tumor depends on its antigenicity and on several other immunomodulatory factors that are produced either by tumor cells or by host cells in the tumor microenvironment. A non-immunogenic tumor can thus not trigger an immune response, regardless the presence or absence of any inhibitory signal sent to immune cells. Non-immunogenic tumors are generally associated with poorer prognoses and hardly respond to most therapeutic strategies.

There is thus a continued and urgent need for novel anticancer therapies and for the identification of new molecules that would allow potentiating the effects of known anticancer treatments.

### SUMMARY OF THE INVENTION

The invention is defined by the claims.

The present inventors have shown that specific benzene sulfonamide thiazole compounds have the ability to remarkably potentiate the effects of anticancer treatments including chemotherapy, targeted therapies and immunotherapies. The specific benzene sulfonamide thiazole compounds used according to the present invention have been disclosed in the international application published under reference WO2014/07248. They have anticancer properties and target GRP78. Surprisingly, the effects obtained when combining these compounds with anticancer treatments is significantly higher than those obtained when using each treatment alone.

The present inventors have further showed that such a combination therapy is triggers remarkable tumor growth inhibition, tumor regression and better overall survival in patients overexpressing either circulating or intratumor GRP78.

Overexpression of GRP78 was shown to be associated with poor prognosis in multiple cancers such as ovarian cancer (see Samanta et al. Scientific reports 10.1 (2020): 1-12), melanoma (Shimizu et al. Pathology & Oncology Research 23.1 (2017): 111-116), lung cancer (Xia et al. Journal of Translational Medicine 19.1 (2021): 1-14), gastric cancer (Zhang et al. Clinical & experimental metastasis 23.7 (2006): 401-410), breast cancer (Oncology letters 10.4 (2015): 2149-2155, esophageal cancer (Zhao et al. Digestive diseases and sciences 60.9 (2015): 2690-2699), pancreatic cancer (Tong et al. Pancreatology 21.7 (2021): 1378-1385) or colorectal cancer (Thornton et al. International journal of cancer 133.6 (2013): 1408-1418). The present inventors have shown that the combination according to the present invention allows significantly reducing the intratumor and circulating levels of GRP78 in patients as compared to a monotherapy comprising either a benzene sulfonamide thiazole compound or said “conventional” anticancer treatment alone.

The combination therapy according to the present invention would thus particularly be beneficial to patients presenting high levels of GRP78 and who are therefore identified as having a poor prognosis.

Accordingly, the present invention pertains to a combination of an anticancer treatment with a benzene sulfonamide thiazole compound of formula (I):

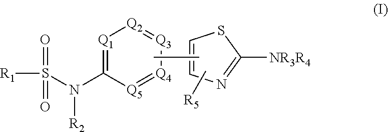

wherein
Q₁ to Q₅ identical or different represent CR₆
R₁ represents C₆-C₁₀ aryl comprising one or two fused rings, wherein from 2 to 5 carbon atoms may be replaced with a heteroatom selected from O, S, N and NR₆, and eventually substituted with from 5 to 11 substituents selected from R₆, halo, CN, NO₂, CF₃, OCF₃, COOR₆, OCOR₆, SO₂NR₆R₇, CONR₆R₇, NR₆R₇, N₆COR₇, (CH₂)ₚ₋ NR₆R₇, (CH₂)ₚ₋ OR₆ and (CH₂)_{P}SR₆,
R₂ is SO₂R₁ or R₆
R₃ and R₄ identical or different are selected from CORs and R₆
R₅ represents R₆, aryl, OR₆, SR₆, halo, CN, NO₂, CF₃, OCF₃, COOR₆, SO₂NR₆R₇, CONR₆R₇, NR₆R₇ and NHCOR₆,
R₆ and R₇ identical or different represent H or alkyl
R₈ is selected from H, alkyl, cycloalkyl, aryl, alkylaryl, wherein aryl may be substituted with from one to four R₅ substituents identical or different,
or R₈ represents —(CH₂)_{q}—NR₆R₇,
p represents an integer from 0 to 6,
q represents an integer from 0 to 6,
wherein the thiazolyl group is linked to the 6-member group in meta or para position with respect to the sulfonamide group and wherein the thiazolyl group is linked to the 6-member group in position α or β with respect to the S atom,
for use in the treatment of cancer in a patient who has been identified as overexpressing GRP78.

### DETAILED DESCRIPTION

Using several cancer mice models (such as a colorectal CT26 allograft model) and in vitro cancer cell-line models (such melanoma, myeloma as well as gastric, pancreatic, colorectal cancer cell lines), the present inventors have shown that a combined therapy using a benzene sulfonamide thiazole compound and a “conventional” anticancer treatment significantly improves the survival rate and strongly reduces tumor growth as compared to monotherapies comprising either a benzene sulfonamide thiazole compound or said “conventional” anticancer treatment alone. They also have shown that the mice treated with the combination significantly decrease the level of circulating and intertumoral GRP78 compared to the vehicle and the “conventional” anticancer treatment

The inventors have notably shown strong synergistic effects between compounds such as immune checkpoint inhibitors and the benzene sulfonamide thiazole compounds disclosed herein. Without wishing to be bound by theory, the present inventors submit that the benzene sulfonamide thiazole compounds described herein render the tumor immunogenic, i.e. detectable by the immune system, thereby allowing for the potentiation of the anticancer effect of the combined anticancer treatment. The inventors have also shown that the specific combination of the benzene sulfonamide thiazole compound with an anticancer treatment is particularly advantageous in patients overexpressing GRP78.

The present invention thus represents an extremely promising therapy for the treatment of cancers.

Accordingly, in a first aspect, the present invention pertains to a combination of an anticancer treatment with a benzene sulfonamide thiazole of formula (I):

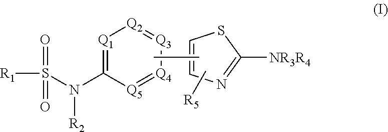

wherein
Q₁ to Q₅ identical or different represent CR₆
R₁ represents C₆-C₁₀ aryl comprising one or two fused rings, wherein from 2 to 5 carbon atoms may be replaced with a heteroatom selected from O, S, N and NR₆, and eventually substituted with from 5 to 11 substituents selected from R₆, halo, CN, NO₂, CF₃, OCF₃, COOR₆, OCOR₆, SO₂NR₆R₇, CONR₆R₇, NR₆R₇, NRCOR₇, (CH₂)ₚ₋ NR₆R₇, (CH₂)ₚ₋ OR₆ and (CH₂)_{P}SR₆,
R₂ is SO₂R₁ or R₆
R₃ and R₄ identical or different are selected from COR₈ and R₆
R₅ represents R₆, aryl, OR₆, SR⁶, halo, CN, NO₂, CF₃, OCF₃, COOR₆, SO₂NR₆R₇, CONR₆R₇, NR₆R₇ and NHCOR₆,
R₆ and R₇ identical or different represent H or alkyl
R₈ is selected from H, alkyl, cycloalkyl, aryl, alkylaryl, wherein aryl may be substituted with from one to four R₅ substituents identical or different,
or R₈ represents —(CH₂)_{q}—NR₆R₇,
p represents an integer from 0 to 6,
q represents an integer from 0 to 6,
wherein the thiazolyl group is linked to the 6-member group in meta or para position with respect to the sulfonamide group and wherein the thiazolyl group is linked to the 6-member group in position α or β with respect to the S atom,
or, if appropriate, their pharmaceutically acceptable salts and/or isomers, tautomers, solvates or isotopic variations thereof
for use in the treatment of cancer in a patient who has been identified as overexpressing GRP78.

The “combination” for use according to the present invention encompasses both:
an anticancer treatment/agent for use in the treatment of cancer, wherein said anticancer agent is used in combination with a benzene sulfonamide thiazole of formula (I) as defined above; and
a benzene sulfonamide thiazole of formula (I) as defined above for use in the treatment of cancer, wherein said benzene sulfonamide thiazole is used in combination with an anticancer agent.

The “combination” for use according to the present invention also encompasses a “kit of parts” comprising:
an anticancer agent; and
a compound of formula (I) as defined above,
   for use in the treatment of cancer in a patient who has been identified as overexpressing GRP78.

The term “kit-of-parts” herein refers to a combined preparation wherein the active ingredients are physically separated for use in a combined therapy by simultaneous administration or sequential administration to the patient. Hence, according to the present invention, the anticancer agent and the compound of formula (I) are administered to the patient in a separate form, either simultaneously, separately or sequentially in any order, for the treatment of cancer. According to a further embodiment, the present invention also pertains to a kit of parts per se, i.e. to a kit of parts comprising:
an anticancer agent; and
a compound of formula (I) as defined above.

### Anticancer Treatment/Agent

According to the present invention, the anticancer treatment is any anticancer agent selected from chemotherapies, targeted therapies, immunotherapies and combinations thereof.

“Chemotherapies”, “chemotherapeutics” and “chemotherapeutic agents” refer to chemical compounds that are effective in inhibiting tumor growth. Examples of chemotherapeutic agents include alkylating agents such as thiotepa and cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethylenethiophosphaoramide and trimethylolomelamine; acetogenins (especially bullatacin and bullatacinone); a camptothecin (including irinotecan and topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CBI-TMI); eleutherobin; pancratistatin; a sarcodictyin; spongistatin; nitrogen mustards such as chlorambucil, chlornaphazine, cholophosphamide, etanautine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, ranimustine; antibiotics such as the enediyne antibiotics (e.g. calicheamicin, especially calicheamicin (11 and calicheamicin 211, see, e.g., Agnew Chem Intl. Ed. Engl. 33:183-186 (1994); dynemicin, including dynemicin A; an esperamicin; as well as neocarzinostatin chromophore and related chromoprotein enediyne antiobiotic chromomophores), aclacinomysins, actinomycin, authramycin, azaserine, bleomycins, cactinomycin, carabicin, canninomycin, carzinophilin, chromomycins, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxydoxorubicin), epirubicin, esorubicin, idanrbicin, marcellomycin, mitomycins, mycophenolic acid, nogalarnycin, olivomycins, peplomycin, potfiromycin, puromycin, quelamycin, rodorubicin, streptomgrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; amsacrine; bestrabucil; bisantrene; edatraxate; defofamine; demecolcine; diaziquone; elfornithine; elliptinium acetate; an epothilone; etoglucid; gallium nitrate; hydroxyurea; lentinan; lonidamine; maytansinoids such as maytansine and ansamitocins; mitoguazone; mitoxantrone; mopidamol; nitracrine; pento statin; phenamet; pirarubicin; podophyllinic acid; 2-ethylhydrazide; procarbazine; PSK®; razoxane; rhizoxin; sizofiran; spirogennanium; tenuazonic acid; triaziquone; 2,2′,2″-trichlorotriethylarnine; trichothecenes (especially T-2 toxin, verracurin A, roridinA and anguidine); urethan; vindesine; dacarbazine; mannomustine; mitobromtol; mitolactol; pipobroman; gacytosine; arabinoside (“Ara-C”); cyclophosphamide; thiotepa; taxoids, e.g. paclitaxel (TAXOL®, Bristol-Myers Squibb Oncology, Princeton, N.].) and docetaxel (TAXOTERE®, Rhone-Poulenc Rorer, Antony, France); chlorambucil; gemcitabine; 6-thioguanine; mercaptopurine; methotrexate; platinum analogs such as cisplatin, carboplatin oxaloplatin; vinblastine; platinum; etoposide (VP-16); ifosfamide; mitomycin C; mitoxantrone; vincristine; vinorelbine; navelbine; novantrone; teniposide; daunomycin; aminopterin; xeloda; ibandronate; CPT-1 1; topoisomerase inhibitor RFS 2000; difluoromethylornithine (DMFO); retinoic acid; capecitabine; and phannaceutically acceptable salts, acids or derivatives of any of the above. Also included in this definition are antihormonal agents that act to regulate or inhibit honnone action on tumors such as anti-estrogens including for example tamoxifen, raloxifene, aromatase inhibiting 4(5)-imidazoles, 4-hydroxytamoxifen, trioxifene, keoxifene, LY117018, onapristone, and toremifene (Fareston); and anti-androgens such as flutamide, nilutamide, bicalutamide, leuprolide, and goserelin; and pharmaceutically acceptable salts, acids or derivatives of any of the above.

According to a preferred embodiment, the chemotherapeutic agent for use in the context of the present invention is selected from doxorubicin, toxoids such as paclitaxel and docetaxel, gemcitabine, anti-metabolites such as methotrexate and 5-fluorouracil (5-FU), platinum analogs such as cisplatin and oxaloplatin, and a camptothecin such as irinotecan and topotecan.

“Targeted therapies” refer to agents that act by blocking the growth of cancer cells by interfering with specific targeted molecules needed for carcinogenesis and tumor growth. Most targeted therapies are either small-molecule drugs or monoclonal antibodies. It is noteworthy that some targeted therapies can qualify as immunotherapeutic agents and/or chemotherapeutic agents. Examples of targeted therapies include Bortezomib, Braf inhibitors such as vemurafenib and dabrafenib, Cobimetinib, Imatinib, Gefitinib, Erlotinib Sorafenib, Sunitinib, Dasatinib, Lapatinib, Nilotinib, tamoxifen, janus kinase inhibitors such as Tofacitinib, ALK inhibitors such as Crizotinib, Bcl-2 inhibitors such as Venetoclax, Obatoclax, navitoclax, and gossypol, PARP inhibitors such as olaparib, rucaparib, niraparib and talazoparib, PI3K inhibitors such as perifosine, Apatinib, Zoptarelin doxorubicin, MEK inhibitors such as trametinib, CDK inhibitors, Hsp90 inhibitors, Hedgehog pathway inhibitors such as vismodegib and sonidegib, salinomycin VAL-083, Vintafolide, Temsirolimus, Everolimus, Vemurafenib, Trametinib, Dabrafenib, monoclonal antibodies including Pembrolizumab, Rituximab, Alemtuzumab, Cetuximab, Panitumumab, Bevacizumab and Ipilimumab.

According to a specific embodiment, the targeted therapy for use according to the present invention is selected from Bortezomib, vemurafenib and Cobimetinib.

“Immunotherapy” “Immunotherapeutics” or “immunotherapeutic agent” refers to a compound, composition or treatment that indirectly or directly enhances, stimulates or increases the body's immune response against cancer cells and/or that decreases the side effects of other anticancer therapies. Immunotherapy is thus a therapy that directly or indirectly stimulates or enhances the immune system's responses to cancer cells and/or lessens the side effects that may have been caused by other anti-cancer agents. Immunotherapy is also referred to in the art as immunologic therapy, biological therapy, biological response modifier therapy and biotherapy. Examples of common immunotherapeutic agents known in the art include, but are not limited to, cytokines, cancer vaccines, monoclonal antibodies and non-cytokine adjuvants. Alternatively, the immunotherapeutic treatment may consist of administering the patient with an amount of immune cells (T cells, NK, cells, dendritic cells, B cells . . . ).

Immunotherapeutic agents can be non-specific, i.e. boost the immune system generally so that the human body becomes more effective in fighting the growth and/or spread of cancer cells, or they can be specific, i.e. targeted to the cancer cells themselves immunotherapy regimens may combine the use of non-specific and specific immunotherapeutic agents.

Non-specific immunotherapeutic agents are substances that stimulate or indirectly improve the immune system. Non-specific immunotherapeutic agents have been used alone as a main therapy for the treatment of cancer, as well as in addition to a main therapy, in which case the non-specific immunotherapeutic agent functions as an adjuvant to enhance the effectiveness of other therapies (e.g. cancer vaccines). Non-specific immunotherapeutic agents can act on key immune system cells and cause secondary responses, such as increased production of cytokines and immunoglobulins. Alternatively, the agents can themselves comprise cytokines. Non-specific immunotherapeutic agents are generally classified as cytokines or non-cytokine adjuvants.

A number of cytokines have found application in the treatment of cancer either as general non-specific immunotherapies designed to boost the immune system, or as adjuvants provided with other therapies. Suitable cytokines include, but are not limited to, interferons, interleukins and colony-stimulating factors.

Interferons (IFNs) include the common types of IFNs, IFN-alpha (IFN-a), IFN-beta (IFN-beta) and IFN-gamma (IFN-y). IFNs can act directly on cancer cells, for example, by slowing their growth, promoting their development into cells with more normal behaviour and/or increasing their production of antigens thus making the cancer cells easier for the immune system to recognise and destroy. IFNs can also act indirectly on cancer cells, for example, by slowing down angiogenesis, boosting the immune system and/or stimulating natural killer (NK) cells, T cells and macrophages. Recombinant IFN-alpha is available commercially as Roferon (Roche Pharmaceuticals) and Intron A (Schering Corporation). The use of IFN-alpha, alone or in combination with other immunotherapeutics or with chemotherapeutics, has shown efficacy in the treatment of various cancers including melanoma (including metastatic melanoma), renal cancer (including metastatic renal cancer), breast cancer, prostate cancer, and cervical cancer (including metastatic cervical cancer).

Interleukins include IL-2, IL-4, IL-11 and IL-12. Examples of commercially available recombinant interleukins include Proleukin® (IL-2; Chiron Corporation) and Neumega® (IL-12; Wyeth Pharmaceuticals). Interleukins, alone or in combination with other immunotherapeutics or with chemotherapeutics, have shown efficacy in the treatment of various cancers including renal cancer (including metastatic renal cancer), melanoma (including metastatic melanoma), ovarian cancer (including recurrent ovarian cancer), cervical cancer (including metastatic cervical cancer), breast cancer, colorectal cancer, lung cancer, brain cancer, and prostate cancer.

Colony-stimulating factors (CSFs) include granulocyte colony stimulating factor (G-CSF or filgrastim), granulocyte-macrophage colony stimulating factor (GM-CSF or sargramostim) and erythropoietin (epoetin alfa, darbepoietin). Various-recombinant colony stimulating factors are available commercially, for example, Neupogen® (G-CSF; Amgen), Neulasta (pelfilgrastim; Amgen), Leukine (GM-CSF; Berlex), Procrit (erythropoietin; Ortho Biotech), Epogen (erythropoietin; Amgen), Arnesp (erytropoietin). Colony stimulating factors have shown efficacy in the treatment of cancer, including melanoma, colorectal cancer (including metastatic colorectal cancer), and lung cancer.

Non-cytokine adjuvants suitable for use in the combinations of the present invention include, but are not limited to, Levamisole, alum hydroxide (alum), Calmette-Guerin bacillus (ACG), incomplete Freund's Adjuvant (IFA), QS-21, DETOX, Keyhole limpet hemocyanin (KLH) and dinitrophenyl (DNP). Non-cytokine adjuvants in combination with other immuno- and/or chemotherapeutics have demonstrated efficacy against various cancers including, for example, colon cancer and colorectal cancer (Levimasole); melanoma (BCG and QS-21); renal cancer and bladder cancer (BCG).

In addition to having specific or non-specific targets, immunotherapeutic agents can be active, i.e. stimulate the body's own immune response, or they can be passive, i.e. comprise immune system components that were generated external to the body.

Active specific immunotherapy typically involves the use of cancer vaccines. Cancer vaccines have been developed that comprise whole cancer cells, parts of cancer cells or one or more antigens derived from cancer cells. Cancer vaccines, alone or in combination with one or more immuno- or chemotherapeutic agents are being investigated in the treatment of several types of cancer including melanoma, renal cancer, ovarian cancer, breast cancer, colorectal cancer, and lung cancer.

The immunotherapeutic treatment may consist of an adoptive immunotherapy as described by Nicholas P. Restifo, Mark E. Dudley and Steven A. Rosenberg “Adoptive immunotherapy for cancer: harnessing the T cell response, Nature Reviews Immunology, Volume 12, April 2012. In adoptive immunotherapy, the patient's circulating lymphocytes, or tumor infiltrated lymphocytes, are isolated in vitro, activated by lymphokines such as IL-2 or transuded with genes for tumor necrosis, and readministered (Rosenberg et al., 1988; 1989). The activated lymphocytes are most preferably the patient's own cells that were earlier isolated from a blood or tumor sample and activated (or “expanded”) in vitro. This form of immunotherapy has produced several cases of regression of melanoma and renal carcinoma.

Passive specific immunotherapy typically involves the use of one or more monoclonal antibodies that are specific for a particular antigen found on the surface of a cancer cell or that are specific for a particular cell growth factor. Monoclonal antibodies may be used in the treatment of cancer in a number of ways, for example, to enhance a subject's immune response to a specific type of cancer, to interfere with the growth of cancer cells by targeting specific cell growth factors, such as those involved in angiogenesis, or by enhancing the delivery of other anticancer agents to cancer cells when linked or conjugated to agents such as chemotherapeutic agents, radioactive particles or toxins.

Monoclonal antibodies currently used as cancer immunotherapeutic agents that are suitable for inclusion in the combinations of the present invention include, but are not limited to, rituximab (Rituxan®), trastuzumab (Herceptin®), ibritumomab tiuxetan (Zevalin®), tositumomab (Bexxar®), cetuximab (C-225, Erbitux®), bevacizumab (Avastin®), gemtuzumab ozogamicin (Mylotarg®), alemtuzumab (Campath®), and BL22. Monoclonal antibodies are used in the treatment of a wide range of cancers including breast cancer (including advanced metastatic breast cancer), colorectal cancer (including advanced and/or metastatic colorectal cancer), ovarian cancer, lung cancer, prostate cancer, cervical cancer, melanoma and brain tumors. Other examples include immune check point inhibitors.

The expression “immune checkpoint protein” is widely known in the art and refers to a molecule that is expressed by T cells and that either turns up a signal (stimulatory checkpoint molecules) or turns down a signal (inhibitory checkpoint molecules). Immune checkpoints constitute immune checkpoint pathways such as the CTLA-4 and PD-1 dependent pathways (see e.g. Pardoll, 2012. Nature Rev Cancer 12:252-264; Mellman et al., 2011. Nature 480:480-489). Examples of inhibitory checkpoint molecules include A2AR, B7-H3, B7-H4, BTLA, CTLA-4, CD277, IDO1, KIR, PD-1, LAG-3, TIM-3 TIGIT and VISTA.

A2AR (the “Adenosine A2A receptor”) is considered as an important checkpoint in cancer therapy: the presence of adenosine in the immune microenvironment leads to an A2a-receptor activation, and induces a negative immune feedback loop and the tumor microenvironment has relatively high concentrations of adenosine. B7-H3, also called CD276, was originally understood to be a co-stimulatory molecule but is now regarded as co-inhibitory. B7-H4, also called VTCN1, is expressed by tumor cells and tumor-associated macrophages and is involved in tumor escape. BTLA (“B and T Lymphocyte Attenuator”), also referred to as CD272, has HVEM (Herpesvirus Entry Mediator) as its ligand. Surface expression of BTLA is gradually downregulated during differentiation of human CD8⁺ T cells from the naive to effector cell phenotype. Tumor specific human CD8⁺ T cells express high levels of BTLA. Expression of CTLA-4 (“Cytotoxic T-Lymphocyte-Associated protein 4”), also called CD152, on Treg cells controls T cell proliferation. IDO1 (“Indoleamine 2,3-dioxygenase 1”) is a tryptophan catabolic enzyme—a immune inhibitory-related enzyme. IDO1 is known to suppress T and NK cells, generate and activate Tregs and myeloid-derived suppressor cells, and promote tumor angiogenesis. KIR (“Killer-cell Immunoglobulin-like Receptor”), is a receptor for MHC Class I molecules on Natural Killer cells. LAG3 (“Lymphocyte Activation Gene-3”) suppresses an immune response via an action on Tregs and a direct inhibitory effect on CD8⁺ T cells. PD-1 (“Programmed Death 1”) receptor, has two ligands, PD-L1 and PD-L2. This checkpoint is the target of pembrolizumab commercialized by Merck. Targeting PD-1 allows restoring immune function in the tumor microenvironment. TIM-3, (“T-cell Immunoglobulin domain and Mucin domain 3”), is expressed on activated human CD4⁺ T cells and regulates Th1 and Th17 cytokines. TIM-3 acts as a negative regulator of Th1/Tc1 function by triggering cell death upon interaction with its ligand, galectin-9. VISTA (“V-domain Ig suppressor of T cell activation”) is primarily expressed on hematopoietic cells. Consistent expression of VISTA on leukocytes within tumors allow VISTA blockade to be effective across a broad range of solid tumors. TIGIT (“T cell immunoreceptor with Ig and ITIM domains”) is an immune receptor present on some percentage of T cells and Natural Killer Cells (NK). TIGIT inhibits T cell activation in vivo.

An “immune checkpoint inhibitor” or “checkpoint blockade cancer immunotherapy agent” has its general meaning in the art and refers to any compound inhibiting the function of an immune inhibitory checkpoint protein. Inhibition includes reduction of function and full blockade. Immune checkpoint inhibitors include peptides, antibodies, nucleic acid molecules and small molecules. Preferred immune checkpoint inhibitors are antibodies that specifically recognize immune checkpoint proteins. The immune checkpoint inhibitor used in the context of the present invention is administered for enhancing the proliferation, migration, persistence and/or cytoxic activity of CD8⁺ T cells in the patient. CD8⁺ T cells are a subset of T cells which express CD8 on their surface. They are MHC class I-restricted, and function as cytotoxic T cells. They are also referred to as cytotoxic T lymphocytes (CTL), T-killer cell, cytolytic T cells, CD8+ T cells or killer T cells. CD8 antigens are members of the immunoglobulin supergene family and are associative recognition elements in major histocompatibility complex class I-restricted interactions. The ability of the immune checkpoint inhibitor to enhance CD8⁺ T cell killing activity may be determined by any assay well known in the art. Typically said assay is an in vitro assay wherein CD8⁺ T cells are brought into contact with target cells (e.g. target cells that are recognized and/or lysed by CD8⁺ T cells). For example, the immune checkpoint inhibitor of the present invention can be selected for its ability to increase specific lysis by CD8⁺ T cells by more than about 20%, preferably with at least about 30%, at least about 40%, at least about 50%, or more. Examples of protocols for classical cytotoxicity assays are conventional.

Typically, the immune checkpoint inhibitor is an agent which blocks an immunosuppressive receptor expressed by activated T lymphocytes, such as cytotoxic T lymphocyte-associated protein 4 (CTLA4) and programmed cell death 1 (PDCD1, also known as PD-1), or by NK cells, like various members of the killer cell immunoglobulin-like receptor (KIR) family, or an agent which blocks the principal ligands of these receptors, such as PD-1 ligand CD274 (best known as PD-L1 or B7-H1).

Typically, the checkpoint blockade cancer immunotherapy agent is an antibody.

In some embodiments, the checkpoint blockade cancer immunotherapy agent is an antibody selected from the group consisting of anti-PD1 antibodies, anti-PDL1 antibodies, anti-PDL2 antibodies, anti-CTLA4 antibodies, anti-TIM-3 antibodies, anti-LAG3 antibodies, anti-IDO1 antibodies, anti-TIGIT antibodies, anti-B7H3 antibodies, anti-B7H4 antibodies, anti-BTLA antibodies, and anti-B7H6 antibodies.

Examples of anti PD-1, anti PD-L1 and anti PD-L2 antibodies are described in U.S. Pat. Nos. 7,488,802; 7,943,743; 8,008,449; 8,168,757; 8,217,149, and PCT Published Patent Application Nos: WO03042402, WO2008156712, WO2010089411, WO2010036959, WO2011066342, WO2011159877, WO2011082400, and WO2011161699. In some embodiments, the PD-1 blockers include anti-PD-L1 antibodies (such as e.g. Atezolizumab, Avelumab or Durvalumab). In other embodiments, the PD-1 blockers include anti-PD-L2 antibodies. In certain other embodiments the PD-1 blockers include anti-PD-1 antibodies and similar binding proteins such as nivolumab (MDX 1106, BMS 936558, ONO 4538), a fully human IgG4 antibody that binds to and blocks the activation of PD-1 by its ligands PD-L1 and PD-L2; lambrolizumab (MK-3475 or SCH 900475), a humanized monoclonal IgG4 antibody against PD-1; CT-011 a humanized antibody that binds PD-1; AMP-224 is a fusion protein of B7-DC; an antibody Fc portion; BMS-936559 (MDX- 1105-01) for PD-L1 (B7-H1) blockade.

Examples of anti-CTLA-4 antibodies are described in U.S. Pat. Nos. 5,811,097; 5,811,097; 5,855,887; 6,051,227; 6,207,157; 6,682,736; 6,984,720; and 7,605,238. One anti-CDLA-4 antibody is tremelimumab, (ticilimumab, CP-675,206). In some embodiments, the anti-CTLA-4 antibody is ipilimumab (also known as 10D1, MDX-DO10) a fully human monoclonal IgG antibody that binds to CTLA-4.

Other immune-checkpoint inhibitors include lymphocyte activation gene-3 (LAG-3) inhibitors, such as IMP321, a soluble Ig fusion protein (Brignone et al., 2007, J. Immunol. 179:4202-4211).

Other immune-checkpoint inhibitors include B7 inhibitors, such as B7-H3 and B7-H4 inhibitors. In particular, the anti-B7-H3 antibody MGA271 (Loo et al., 2012, Clin. Cancer Res. July 15 (18) 3834).

Also included are TIM3 (“T-cell immunoglobulin domain and mucin domain 3”) inhibitors (Fourcade et al., 2010, J. Exp. Med. 207:2175-86 and Sakuishi et al., 2010, J. Exp. Med. 207:2187-94). The natural ligand of TIM-3 is galectin 9 (Gal9). Accordingly, the term “TIM-3 inhibitor” as used herein refers to a compound, substance or composition that can inhibit the function of TIM-3. For example, the inhibitor can inhibit the expression or activity of TIM-3, modulate or block the TIM-3 signaling pathway and/or block the binding of TIM-3 to galectin-9. Antibodies having specificity for TIM-3 are well known in the art and typically those described in WO2011155607, WO2013006490 and WO2010117057.

In some embodiments, the immune checkpoint inhibitor is an Indoleamine 2,3-dioxygenase (IDO) inhibitor, preferably an IDO1 inhibitor. Examples of IDO inhibitors are described in WO 2014150677. Examples of IDO inhibitors include without limitation 1-methyl-tryptophan (IMT), β-(3-benzofuranyl)-alanine, β-(3-benzo(b)thienyl)-alanine), 6-nitro-tryptophan, 6-fluoro-tryptophan, 4-methyl-tryptophan, 5-methyl tryptophan, 6-methyl-tryptophan, 5-methoxy-tryptophan, 5-hydroxy-tryptophan, indole 3-carbinol, 3,3′-diindolylmethane, epigallocatechin gallate, 5-Br-4-Cl-indoxyl 1,3-diacetate, 9-vinylcarbazole, acemetacin, 5-bromo-tryptophan, 5-bromoindoxyl diacetate, 3-Amino-naphtoic acid, pyrrolidine dithiocarbamate, 4-phenylimidazole a brassinin derivative, a thiohydantoin derivative, a β-carboline derivative or a brassilexin derivative. Preferably the IDO inhibitor is selected from 1-methyl-tryptophan, β-(3-benzofuranyl)-alanine, 6-nitro-L-tryptophan, 3-Amino-naphtoic acid and β-[3-benzo(b)thienyl]-alanine or a derivative or prodrug thereof.

In some embodiments, the immune checkpoint inhibitor is an anti-TIGIT (T cell immunoglobin and ITIM domain) antibody.

According to a specific embodiment, the anticancer treatment according to the present 20 invention is an immune checkpoint inhibitor, preferably selected from an anti-PD-1 antibody and an anti-PD-L1 antibody.

In a preferred embodiment, the checkpoint blockade cancer immunotherapy agent is a PD-1 blocking antibody, such as Nivolumab or Pembrolizumab.

### Compounds of Formula (I)

The benzene sulfonamide thiazole compounds of formula (I) used in the context of the present invention have been extensively disclosed and studied in the international patent application published under reference WO2014/072486, the content of which is hereby incorporated by reference in its entirety.

According to a preferred embodiment, the compounds of formula (I) used in the context 30 of the present invention also fall under formula (II):

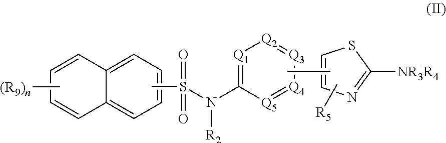

wherein Q₁ to Q₅, R₂, R₃, R₄ and R₅ are as defined above, and R₉ represents R₆, halo, CN, NO₂, CF₃, OCF₃, COOR₆, OCOR₆, SO₂NR₆R₇, CONR₆R₇, NR₆R₇, N₆COR₇, (CH₂)ₚ₋ NR₆R₇, (CH₂)ₚ₋ OR₆ and (CH₂)_{P}SR₆.
wherein R₆, R₇ and p are as defined above and n represents 1, 2, 3 or 4 and the naphtyl group is attached to the sulfur atom in position 1, 2 or 3 with respect to the quaternary carbons.

In the above general formulae (I) or (II):
R₂ preferably represents H,
R₃ preferably represents H,
R₄ is preferably a CO-alkyl,
R₆ is preferably H or alkyl,
R₉ is preferably NR₆R₇, wherein R₆ and R₇ both preferably represent CH₃,
the thiazolyl group is preferably in the meta position with respect to the sulfonamide group and is also preferably linked to the 6 member aromatic ring in the P-position with respect to the sulfur atom.

In the above general formulae (I) and (II), alkyl denotes a straight-chain or branched group containing 1, 2, 3, 4 or 5 carbon atoms. This also applies if they carry substituents or occur as substituents of other radicals, for example in O-alkyl radicals, S-alkyl radicals etc. Examples of suitable alkyl radicals are methyl, ethyl, n-propyl, isopropyl, n-butyl, isobutyl, sec-butyl, tert-butyl, etc.

Cycloalkyl comprises 3 to 7 carbon atoms, for example cyclopropyl, cyclobutyl, cyclopentyl, cyclohexyl and cycloheptyl.

Aryl denotes an aromatic carbon ring comprising from 6 to 10 carbon atoms.

Finally, halo denotes a halogen atom selected from the group consisting of fluoro, chloro, bromo and iodo in particular fluoro or chloro.

Preferably, in Formulae (I) and (II), the free bond on the phenyl group means that the phenyl can be substituted in the meta or para position.

Preferred compounds according to the invention are:
N-(4-(3-(5-(dimethylamino)naphthalene-1-sulfonamido)phenyl)thiazol-2-yl)acetamide 5-(dimethylamino)-N-(3-(2-(methylamino)thiazol-4-yl)phenyl)naphthalene-1-sulfonamide
N-(4-(3-(5-(dimethylamino)naphthalene-1-sulfonamido)phenyl)thiazol-2-yl)-4
methylbenzamide
N-(3-(2-aminothiazol-4-yl)phenyl)-5-(dimethylamino)naphthalene-1-sulfonamide
N-(4-(3-(5-(dimethylamino)naphthalene-1-sulfonamido)phenyl)thiazol-2-yl)benzamide
N-(4-(3-(5-(dimethylamino)naphthalene-1-sulfonamido)phenyl)thiazol-2-yl)pivalamide
2-fluoro-N-(3-(2-(methylamino)thiazol-4-yl)phenyl)benzenesulfonamide
N-(4-(4-(naphthalene-2-sulfonamido)phenyl)thiazol-2-yl)acetamide
N-(4-(4-(5-(dimethylamino)naphthalene-1-sulfonamido)phenyl)thiazol-2-yl)acetamide
N-(4-(4-(2-fluorophenylsulfonamido)phenyl)thiazol-2-yl)acetamide
N-(4-(4-(2,4-difluorophenylsulfonamido)phenyl)thiazol-2-yl)acetamide
N-(4-(4-(3-(trifluoromethyl)phenylsulfonamido)phenyl)thiazol-2-yl)acetamide
N-(4-(3-(3-(trifluoromethyl)phenylsulfonamido)phenyl)thiazol-2-yl)acetamide
N-(4-(4-(4-(trifluoromethyl)phenylsulfonamido)phenyl)thiazol-2-yl)acetamide
N-(4-(3-(4-methylphenylsulfonamido)phenyl)thiazol-2-yl)acetamide
N-(4-(3-(2-nitrophenylsulfonamido)phenyl)thiazol-2-yl)acetamide
N-(4-(3-(3-nitrophenylsulfonamido) phenyl)thiazol-2-yl)acetamide
N-(4-(3-(phenylsulfonamido)phenyl)thiazol-2-yl)acetamide
N-(4-(3-(methylsulfonamido)phenyl)thiazol-2-yl)acetamide
N-(4-(4-(4-methylphenylsulfonamido) phenyl)thiazol-2-yl)acetamide and
N-(4-(3-(5-(dimethylamino)naphthalene-1-sulfonamido)phenyl)thiazol-2-yl)-6-amino-hexanamide.

According to a more preferred embodiment, the benzene sulfonamide thiazole compound of formula I used in the context of the present invention corresponds to formula (III):

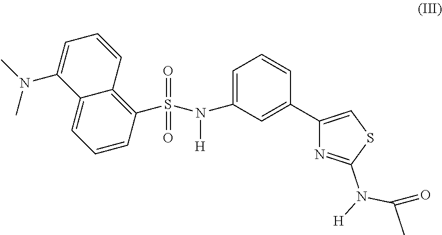 [SMILES: CC(=O)Nc1nc(-c2cccc(NS(=O)(=O)c3cccc4c(N(C)C)cccc34)c2)cs1]

The compound of Formula (III) is also referred to as compound “HA15” in the context of the present invention.

Methods for synthetizing the benzene sulfonamide thiazole compounds useful according to the present invention are disclosed in application WO2014/072486 mentioned above.

The benzene sulfonamide thiazole compounds of formulae (I), (II) and (III) can be in the form of pharmaceutically acceptable salts. Pharmaceutically acceptable salts include the acid addition and base salts thereof.

Suitable acid addition salts are formed from acids, which form non-toxic salts. Examples include the acetate, aspartate, benzoate, besylate, bicarbonate/carbonate, bisulphate/sulphate, borate, camsylate, citrate, edisylate, esylate, formate, fumarate, gluceptate, gluconate, glucuronate, hexafluorophosphate, hibenzate, hydrochloride/chloride, hydrobromide/bromide, hydroiodide/iodide, isethionate, lactate, malate, maleate, malonate, mesylate, methylsulphate, naphthylate, 2-napsylate, nicotinate, nitrate, orotate, oxalate, palmitate, pamoate, phosphate/hydrogen phosphate/dihydrogen phosphate, saccharate, stearate, succinate, tartrate, tosylate and trifluoroacetate and xinafoate salts.

Suitable base salts are formed from bases which form non-toxic salts. Examples include the aluminium, arginine, benzathine, calcium, choline, diethylamine, diolamine, glycine, lysine, magnesium, meglumine, olamine, potassium, sodium, tromethamine and zinc salts. Hemisalts of acids and bases may also be formed, for example, hemisulphate and hemicalcium salts. For a review on suitable salts, see “Handbook of Pharmaceutical Salts: Properties, Selection, and Use” by Stahl and Wermuth (Wiley- VCH, Weinheim, Germany, 2002).

The benzene sulfonamide thiazole compounds used in the context of the present invention may exist in both unsolvated and solvated forms. The term ‘solvate’ describes a molecular complex comprising the benzene sulfonamide thiazole compound and a stoichiometric amount of one or more pharmaceutically acceptable solvent molecules, for example, ethanol. The term ‘hydrate’ is employed when said solvent is water. Also included are complexes such as clathrates, drug-host inclusion complexes wherein, in contrast to the aforementioned solvates, the drug and host are present in stoichiometric or non-stoichiometric amounts. Also included are complexes of the drug containing two or more organic and/or inorganic components, which may be in stoichiometric or non-stoichiometric amounts. The resulting complexes may be ionised, partially ionized, or non-ionized. For a review of such complexes, see J Pharm Sci, 64 (8), 1269-1288 by Haleblian (August 1975).

The benzene sulfonamide thiazole compounds of formula (I) thus include references to salts, solvates and complexes thereof and to solvates and complexes of salts thereof. The benzene sulfonamide thiazole compounds of formula (I) include all polymorphs and crystal habits thereof, prodrugs and isomers thereof (including optical, geometric and tautomeric isomers) and isotopically- labeled compounds of formula (I).

### Therapeutic Use

As mentioned above, the anticancer treatment and the benzene sulfonamide thiazole compound according to the present invention are used for the treatment of cancer in a patient.

The present disclosure thus provides a method for treating cancer comprising administering, to a patient in need thereof, a therapeutically effective amount of an anticancer treatment and of the benzene sulfonamide thiazole as defined above.

According to the present invention, the anticancer treatment and the benzene sulfonamide thiazole compound are administered to the patient either simultaneously, separately or sequentially in any order. According to a specific embodiment, the anticancer treatment is administered after the benzene sulfonamide thiazole compound. In other words, the anticancer treatment is administered to a patient who has already received the benzene sulfonamide thiazole compound. According to another specific embodiment, the benzene sulfonamide thiazole compound is administered after the anticancer treatment. In other words, the benzene sulfonamide thiazole compound is administered to a patient who has already received the immune checkpoint inhibitor.

The terms “Subject” and “Patient” refer to a human or an animal suffering from cancer. Typically, the patient is a mammal. The patient can e.g. be a human, a feline such as a cat, a canine such as a dog or an equid such as a horse. Preferably, the patient is human.

The patient according to the present invention overexpresses GRP78.

“78 kDa glucose-regulated protein” or “GRP78”, also known as “Binding immunoglobulin protein” (BiP) or “heat shock 70 kDa protein 5” (HSPA5) is a protein that in humans is encoded by the HSPA5 gene. It is an endoplasmic reticulum chaperone that plays a key role in protein folding and quality control in the endoplasmic reticulum lumen. The sequence of GRP78 is available under reference P11021 in the Uniprot database library.

By “overexpresses” it is meant that the level of GRP78 measured in a biological sample obtained from the patient is significantly higher than the level measured in a control sample. Typically, said control sample can be a sample obtained from a control population or from a control tissue. A control population is typically constituted of healthy subjects, i.e. subjects who do not suffer from cancer or any other disease. A control tissue is typically constituted of healthy tissues, i.e. tissues not affected by a disease. Said control tissue is usually obtained from the patient himself. The skilled person knows several techniques that allow determining whether a gene/protein is overexpressed as compared to a control sample. The skilled person is familiar with such techniques which are used routinely. Several studies have been published with respect to the evaluation of GRP78 levels (see e.g. Huang et al (2018). International Journal of Clinical and Experimental Pathology, 11(11), 5223). Typically, the level of GRP78 is considered as “overexpressed” when said level is 1.5, preferably 1.7, more preferably 2 times higher than the level measured in a control population.

Typically, the GRP78 level measured is either the circulating GRP78 level or the intratumor GRP78 level.

“Circulating GRP78” refers to the GRP78 protein present in the blood circulation of the patient, typically in the cell-free section of the blood (plasma/serum) of the patient. The level of circulating GRP78 protein is typically measured by evaluating the quantity of GRP78 in a blood sample, typically a plasma/serum sample obtained from the patient. The control used for determining whether a level of circulating GRP78 is overexpressed is typically the GRP78 level measured in blood samples from a control population.

“Intratumor GRP78” refers to the GRP78 protein expressed within the tumor of the patient. The level of the GRP78 protein is typically measured by evaluating the quantity of GRP78 expressed in a tumor sample, typically in a tumor biopsy obtained from the patient. The control used for determining whether the level of intratumor GRP78 is overexpressed is typically the GRP78 level measured in control tissues, i.e. healthy tissues from the patient.

The person skilled in the art is familiar with many techniques that are used on a daily basis to determine the expression level of a protein such as GRP78.

Such methods typically involve contacting the biological sample to be analyzed with an agent capable of specifically binding the target protein. This agent is usually a polyclonal or monoclonal antibody. The presence of the protein is then typically detected by standard immunodetection methods after separation of the proteins by electrophoresis (technique also called “Western blotting”) or by immunoassays by direct, indirect, competition or immunocapture methods (techniques also called “ELISA”). The formation of a complex between the protein of interest and the antibody(s) targeting said protein is usually detected and quantified by measuring an enzymatic reaction generating a colored, chemiluminescent or fluorescent product leading in a specific staining pattern with a staining %. Typically “strong staining” is defined as ≥50% of the tumor cells staining positive, “moderate staining” is defined as 10 to <50% of the tumor cells staining positive, and “weak staining” is defined as <10% of the tumor cells staining positive (Samanta et al, 2022).

Several kits are currently available for measuring the plasma/serum level GRP78 level. For instance, the ELISA kit commercialized by Enzo Life Sciences under reference ADI-900-214 can be used.

It is also possible to determine the intratumor GRP78 level by determining the density of cells expressing GRP78. Typically, methods for measuring the density GRP78 expressing cells comprise a step of contacting the tumor tissue sample with at least one selective binding agent capable of selectively interacting with GRP78. The selective binding agent may be a polyclonal antibody or a monoclonal antibody, an antibody fragment, synthetic antibodies, or other protein-specific agents such as nucleic acid or peptide aptamers. The skilled person knows several antibodies which are specific to GRP78. Many of these antibodies are commercially available. Immunohistochemistry is particularly suitable for evaluating the density of GRP78 cells. Typically, the tissue tumor sample is firstly incubated with labelled antibodies directed against GRP78. After washing, the labelled antibodies which are bound to GRP78 are revealed by the appropriate technique, depending of the kind of label born by the labelled antibody, e.g. radioactive, fluorescent or enzyme label.

The level of GRP78 can also be measured by determining the quantity of mRNA produced by the HSPA5 gene. Methods for determining a quantity of mRNA are well known in the art. For example, nucleic acid contained in the samples is first extracted according to standard methods, for example using lytic enzymes or chemical solutions or extracted by nucleic-acid-binding resins following the manufacturer's instructions. The extracted mRNA is then detected by hybridization (e. g., Northern blot analysis) and/or amplification (e.g., RT-PCR). Quantitative or semi-quantitative RT-PCR are preferred methods.

Accordingly, according to a specific embodiment, the present disclosure provides a method for treating cancer in a patient comprising a step of measuring the level of GRP78 in a tumor or blood/plasma/serum sample obtained from said patient, and then a step of administering a therapeutically effective amount of an anticancer treatment and of the benzene sulfonamide thiazole as defined above if said patient is identified as overexpressing GRP78.

For the avoidance of doubt, references herein to “treatment” include references to curative, palliative and prophylactic treatment. A “treatment” aims at reversing, alleviating, inhibiting the progress of, or preventing the disorder or condition to which such term applies, or reversing, alleviating, inhibiting the progress of, or preventing one or more symptoms of the disorder or condition to which such term applies.

The term “cancer” herein refers to the physiological condition in subjects that is characterized by unregulated or dysregulated cell growth or death. The term “cancer” includes solid tumors and blood born tumors.

Typically, the combination for use according to the present invention applies to various organs of cancer origin (such as breast, colon, gastric, rectum, pancreatic, lung, skin, head and neck, bladder, ovary, prostate), and also to various cancer cell types (adenocarcinoma, squamous cell carcinoma, large cell cancer, melanoma, etc).

In a particular embodiment, the patient suffers from a solid cancer selected from the group consisting of skin cancer (e.g. melanoma, nonmelanoma skin cancer), colorectal cancer, adrenal cortical cancer, anal cancer, bile duct cancer (e.g. periphilar cancer, distal bile duct cancer, intrahepatic bile duct cancer), bladder cancer, bone cancer (e.g. osteoblastoma, osteosarcoma, chondrosarcoma, fibrosarcoma, malignant fibrous histiocytoma), sarcomas such as liposarcoma and soft-tissue sarcoma, brain and central nervous system cancer (e.g. meningioma, astocytoma, oligodendrogliomas, ependymoma, gliomas, medulloblastoma, ganglioglioma, Schwannoma, germinoma, craniopharyngioma), breast cancer (e.g. ductal carcinoma in situ, infiltrating ductal carcinoma, infiltrating lobular carcinoma, lobular carcinoma in situ), cervical cancer, endometrial cancer (e.g. endometrial adenocarcinoma, adenocanthoma, papillary serous adnocarcinoma), esophagus cancer, gallbladder cancer (mucinous adenocarcinoma, small cell carcinoma), gastrointestinal carcinoid tumors (e.g. choriocarcinoma, chorioadenoma destruens), kidney cancer (e.g. renal cell cancer), laryngeal and hypopharyngeal cancer, liver cancer (e.g. hepatic adenoma, hepatocellular carcinoma), lung cancer (e.g. small cell lung cancer, non-small cell lung cancer), mesothelioma, nasal cavity and paranasal sinus cancer (e.g. esthesioneuroblastoma, midline granuloma), nasopharyngeal cancer, neuroblastoma, oral cavity and oropharyngeal cancer, ovarian cancer, pancreatic cancer, penile cancer, pituitary cancer, prostate cancer, retinoblastoma, rhabdomyosarcoma (e.g. embryonal rhabdomyosarcoma, alveolar rhabdomyosarcoma, pleomorphic rhabdomyosarcoma), salivary gland cancer, stomach cancer, testicular cancer (e.g. seminoma, nonseminoma germ cell cancer), thymus cancer, thyroid cancer (e.g. follicular carcinoma, anaplastic carcinoma, poorly differentiated carcinoma, medullary thyroid carcinoma), vaginal cancer, vulvar cancer, and uterine cancer (e.g. uterine leiomyosarcoma).

In a particular embodiment, the patient suffers from a hematological cancer such as leukaemia, lymphoma (such as Hodgkin lymphoma or non-Hodgkin lymphoma) and myeloma.

The benzene sulfonamide thiazole compounds according to the present invention have been shown to have high potency against both sensitive and resistant cancer cell lines from melanoma, pancreatic cancer, and chronic myeloid leukemia (see Millet et al., Journal of medicinal chemistry 59.18 (2016): 8276-8292). Thus, according to another specific embodiment, the cancer treated according to the present invention is selected from the group consisting of skin cancer, pancreatic cancer and leukemia.

According to a preferred embodiment, the cancer treated according to the present invention is a colorectal cancer, a gastric cancer, a pancreatic cancer, a breast cancer, a lung cancer or skin cancer (preferably melanoma).

According to a specific embodiment, the cancer according to the present invention is a non-immunogenic tumor. By “non-immunogenic tumor” it is herein referred to a tumor that does not elicit T-cell response. The skilled person is familiar with this notion and knows how to determine whether a tumor is immunogenic or not (see e.g. Wang et al. Eife 8 (2019): e49020). Such tumors are generally associated with:
few tumor-infiltrating lymphocytes, and, accordingly with a low density of tumor-infiltrating lymphocytes, either at the margin or in the center of the tumor as e.g. determined in the international application published under reference WO2007/045996 or according to the Immunoscore®;
an exhausted population of CD8⁺ T cells—also referred to as “Tex cells” (see Wherry, E. John, and Makoto Kurachi, Nature Reviews Immunology 15.8 (2015): 486-499) which can be identified as shown in Bengsch et al. Immunity 48.5 (2018): 1029-1045;
limited tumor antigen presentation (as shown in Wang (2019));
a majority of M2 macrophages and myeloid-derived suppressor cells; and/or
type-2 inflamed tumor microenvironment (see e.g. Gajewski et al. Tumor immune microenvironment in cancer progression and cancer therapy (2017): 19-31 or Trujillo et al. Cancer immunology research 6.9 (2018): 990-1000).

According to another embodiment, the cancer according to the present invention is “resistant” to immunotherapy meaning that the patient does not or poorly respond to immunotherapy, and particularly to an immune checkpoint inhibitor monotherapy.

As used herein, the term “responder” refers to a patient that will achieve a response, i.e. a patient where the cancer is eradicated, reduced or stabilized after treatment. A non-responder or refractory patient includes patients for whom the cancer does not show reduction or stabilization after the immunotherapy, and particularly after the immune checkpoint therapy.

The compounds used in the context of the present invention may be administered by any suitable route. The skilled person knows which route of administration to use as well as the corresponding dosages. Compounds useful in the context of the present invention are typically administered via parenteral (e.g., intravenous, intramuscular or subcutaneous) or via oral administration.

The invention will be further illustrated by the following figures and examples. However, these examples and figures should not be interpreted in any way as limiting the scope of the present invention.

### BRIEF DESCRIPTION OF THE FIGURES

FIG. 1: CT26 cells (5.104) are injected subcutaneously in Balb/C mice. As soon as the tumors reach 50 mm³, mice are randomly distributed into different experimental groups and treated daily (intraperitoneal) with Vehicle or HA15 (35 mg/kg) and twice a week during two weeks with anti-PD1 (6.25 mg/kg) or PBS. A: Individual tumor growth curves. B: Survival curves. Mice are euthanized when their tumor reach 800 mm³. A log-rang (Mantel Cox) test was performed to compare statistically the survival in the different experimental groups. Ns=non-significant, *: p≤0.05, **: p≤0.01, ***: p≤0.001.

FIG. 2: HA15 induces in vivo the expression of CHOP and IFNg in mice tumors: tumors were harvested from the mice after HA15 treatment and crushed into lysis buffer to get protein lysates. (A). Tumor lysates were analyzed by western blotting with CHOP, IFNγ and HSP90 antibodies. Densitometric analyses were then performed on the radiogram presented here thanks to the ImageJ (Fiji) program. The signal intensity of the target protein is weighted by that of the HSP90 control. (B) The intra-tumoral IFN-7 was dosed in the lysates by a Mouse IFN-g ELISA kit. Mann-Whitney test was performed to compare statistically the protein expressions in the two different groups.

FIG. 3: Graph showing the variation of the intratumor GRP78 level measured in mice treated with the combination therapy according to the invention as compared to monotherapies comprising either HA15 (BPR001) or an anti-PDL1 alone.

### EXAMPLES

### Example 1: Combination of HA15 and an Anti-PD1 Antibody in a CT26 Tumor Model

### Material and Methods

CT26 Allografts Experiment Female BALB/cOlaHsd mice were obtained at 5 weeks of age from Envigo Laboratory. One week later, these mice were inoculated subcutaneously with CT26 cells (5×10⁵ cells/mouse). As soon as its tumor reach 50 mm³, each mouse was allocated in the different experimental group following a randomization table created with the “Graphpad Quickcalcs” tool (7 mice/group). Since then, animals received intraperitoneal injection of DMA/Tween80/Labrafil (9/1/90)+PBS [Control group], HA15 in Labrafil (0.7 mg/mouse/day)+PBS [HA15 group], anti-PD1 (InvivoMAb anti-mouse PD1 (CD279) antibody/Catalog BE0146/Clone: RPM1-14) in PBS (4 injections of 0.125 mg/mouse at Day 1, 4, 8 and 11 of treatment)+Labrafil [anti-PD1 group] or the combination of the HA15 and the anti-PD1 treatment [HA15+anti-PD1 group]. Tumor volumes were determined using the equation Volume=(Length×(Width)²)/2. When the tumor reached 800 mm³, mice were anesthetized by Ketamine (100 mg/kg)/Xylasine (10 mg/kg) injection to get a retro-orbital blood sample. After sacrifice of the mouse by cervical dislocation, lymph nodes and tumors were harvested for western blot and ELISA experiments.

The survival curve represents the percentage of the mice whose tumors reach 800 mm³ (=death event) through the time. A log-rang (Mantel Cox) test was performed to compare statistically the survival in the different experimental groups. Ns=non-significant, * p≤0.05, **: p≤0.01, ***: p≤0.001

### Intra-Tumoral Proteins Analysis by Western Blot and ELISA.

Pieces of tumor were crushed thanks to the “Precellys 24 Tissue Homogenizer” into CK Mix Tubes (#P000918-LYSK0, Bertin Technologies) in presence of protein lysis buffer containing 50 mmol/l Tris-HCl (pH 7.5), 15 mmol/l, NaCl, 1% Triton X-100, and 1× protease and phosphatase inhibitors. Tumor lysates were then centrifuged to remove cells debris.

For western Blot, tumor lysates are prepared in Laemmli buffer. Briefly, tumor lysates (30 μg) were separated by SDS-PAGE, transferred onto a polyvinylidene fluoride membrane (Millipore), and then exposed to the appropriate antibodies. The antibody directed against IFN-gamma was purchased from Abcam (ab133566). Proteins were visualized with the ECL System from Amersham. The western blot analyses shown are representative of at least three independent experiments.

The intra-tumoral IFN-gamma was dosed in the same lysates by the Mouse Interferon-g ELISA kit from Cusabio (#CSB-E04578m). Briefly, 75 μg tumor lysates (or standards samples) were added to the plate pre-coated with an antibody specific for IFN-γ. After removing any unbound substances, a biotin-conjugated antibody specific for IFN-γ was added to the wells. Then, following an avidin conjugated Horseradish Peroxidase (HRP) incubation, the substrate solution was added. Color developed in proportion to the amount of IFN-γ bound in the initial step. The statistical analysis was performed by a Mann-Whitney test. Ns=non-significant, *: p≤0.05, **: p≤0.01, ***: p≤0.001 Serum Proteins analysis by ELISA.

The mice's blood (500 μL) was placed directly on tube containing a coagulation activator (Microvette 500ZGel #20-1344, Sarstedt) and centrifuge 5 minutes at 10000 g to collect the serum. The serum IFN-gamma was dosed into these samples (100 μL) by the Mouse Interferon-g ELISA kit from Cusabio (#CSB-E04578m) like described above. The statistical analysis was performed by a Mann-Whitney test. Ns=non-significant, * p≤0.05, ** p≤0.01, *** p≤00.001.

### Results:

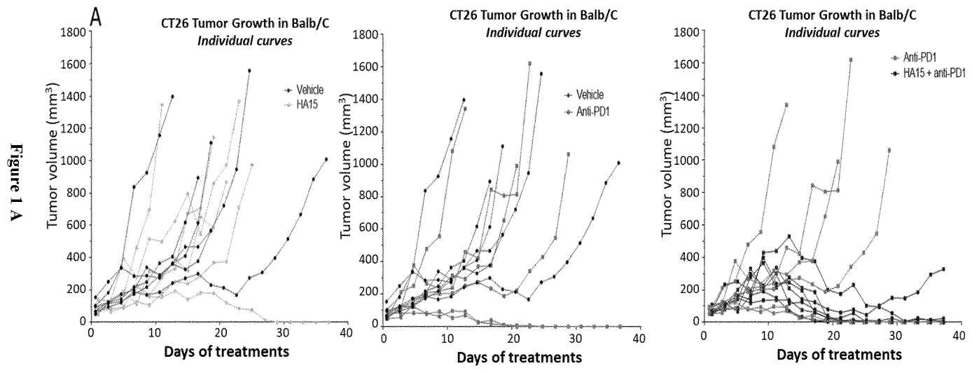

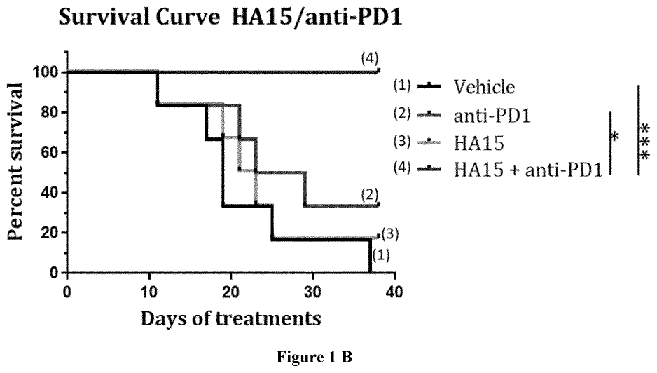

As shown in FIG. 1, a combined therapy using HA15 and an anti-PD-1 antibody significantly improved survival and reduced tumor growth in colorectal cancer mice syngeneic immune-competent models as compared to either a single HA15 (14% complete response) or single anti-PD-1 (33% complete response) antibody treatment. Combination HA15 with anti-PD-1 immunotherapy (Anti PD-1) has led to 100% survival at 40 days.

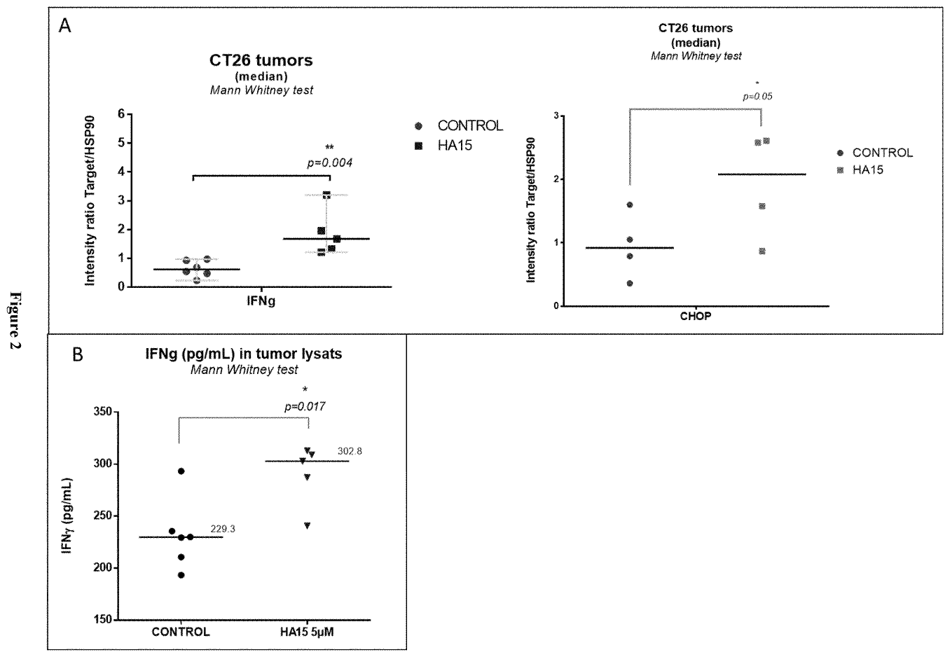

The inventors have further shown that HA15 induces CHOP and IFNγ expressions in CT26 tumors (see FIG. 2), showing an ER (endoplasmic reticulum) stress modulation mediated action. This means that HA15 is able to re-activate CD8+ T cells lymphocyte through its mode of action and subsequent IFNγ release.

The inventors have further shown that immune stimulation is localized to the tumor and does not affect liver in which no variation of the expression of CHOP or IFNγ is observed after HA15 treatment (data not shown).

These results show that HA15 allows potentiating the anti-PD1 response. These results also show that HA15 can be used for turning non-immunogenic tumors into immunogenic tumors susceptible to treatment with e.g. immune checkpoint inhibitors.

### Example 2: Combination of HA15 and an Anti-PDL1 Antibody in a CT26 Tumor Model

### A—Protocol

Mice: A total of 60 female BALB/C mice aged 5 weeks and weighted between 15-19 g, were ordered from Envigo. These had one week of acclimatation in C3M animal facility. Animals were housed in IVC cages (5 per cage) with individual mice identified by ear tag. All animals were allowed free access to a standard certified commercial diet and sanitised water during the study. The holding room was maintained under standard conditions: 18-24° C., 55-70% humidity and a 12h light/dark cycle. One week before dosing start, 2 animals died while blood sampling.

All protocols used in this study have been approved by C3M's committee of Welfare and Ethical.

58 Mice were randomly assigned into the following treatment groups:

| | | | | Dosing | Dosing |
|---|---|---|---|---|---|
| Group | N | Treatment | Dose | route | schedule |
| 1 | 9 | Vehicle + | 100 μg/mouse/ | PO + IP | QD + BIW |
| | | IgG2b | injection | | |
| 2 | 9 | HA15 + | 50 mg/kg + | PO + IP | QD + BIW |
| | | IgG2b | 100 μg/mouse | | |
| 3 | 10 | HA15 + | 150 mg/kg + | PO + IP | QD + BIW |
| | | IgG2b | 100 μg/mouse | | |
| 4 | 10 | HA15 + | 320 mg/kg + | PO + IP | QD + BIW |
| | | IgG2b | 100 μg/mouse | | |
| 5 | 10 | Vehicle + | 100 μg/mouse/ | PO + IP | QD + BIW |
| | | αPDL1 | injection | | |
| 6 | 10 | HA15 + | 320 mg/kg + | PO + IP | QD + BIW |
| | | αPDL1 | 100 μg/mouse | | |

Cells: CT26 cell lines were thawed 2 weeks before mice arrival. These were cultured in RPMI medium supplemented with 10% FBS, 1% Penicillin/streptomycin and 2% sodium pyruvate. Cells were expanded 48 hrs prior to the subcutaneous injection in 5×T175 flasks at concentration of 0.8×10⁶ cells. This concentration allows cells to be 70-80% confluency at the day of the injection. Briefly, cells were washed with PBS, detached from the flasks using 3 ml of trypsin, collected with RPMI medium and centrifuged at 300 g for 5 min. These were then resuspended in PBS, counted and 0.5×10⁶ cells/mouse (32 million cells in total for 80 mice) were taken and re-centrifuged to be resuspended in 100 μl/mouse of PBS ready to be subcutaneously injected (8 ml in total for 80 mice).

### Formulation:

HA15: 15% Kolliphor H15, 100/PEG400, 5% Ethanol, 700/ultrapure water.

1—In a sterile biosafety cabinet, Kolliphor HS15, PEG400 and ethanol were mixed in the appropriate fractions (3/2/1, v/v/v) prior to formulating the compound (Kolliphor HS15 was melt at 30° C.).

2—The appropriate amount of HA15 compound was weighted on a microbalance into a wheaton vial. The compound was dissolved in 3 parts of the final vehicle volume with the above solution using a magnetic bar.

3—After complete solubilization, ultrapure water (7 parts of the final vehicle volume), was added to the wheaton vial and mixed well by vortexing.

Antibodies (antiPDL1 and IgG2b) were diluted in PBS prior to IP injection.

### B—Results

After 13 days of treatment, data showed that daily oral administration of HA15 decreased tumor volume. This reduction in CT26 tumor growth is statistically significant compared with vehicle group. Moreover, this was confirmed after mice termination at day 13 by weighting the tumors.

Anti-PDL1 bi-weekly dosing together with daily administration of HA15 significantly decreased CT26 tumors compared to vehicle+IgG2b control group and mono-treatment (HA15) group.

These results show that HA15 also allows potentiating the anti-PDL1 response. It can therefore be concluded that HA15 allows potentiating the effects of immune checkpoint inhibitors in general.

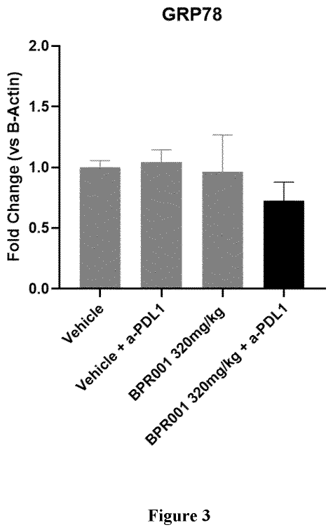

The GRP78 intratumor level of the treated mice is shown in FIG. 3. As shown in this figure, the combination therapy HA15+anti-PDL1 allows significantly reducing the intratumor level of GRP78 in patients as compared to a monotherapy comprising either HA15 or an anti-PDL1 alone

### Example 3: Combination of HA15 and Different Anticancer Treatments

### Methods

Cell lines and reagents: The different cell lines were purchased from ATCC. The tumor cell lines were maintained at 37C° and 5% CO2 in humidified atmosphere and grown in DMEM, high glucose, GlutaMAX™ Supplement, pyruvate growth media supplemented with 10% of fetal bovine serum, (ThermoFisher). Cells were treated with the indicated anticancer agent and HA15 at the indicated concentrations and time. All drugs were dissolved in DMSO.

Proliferation analysis: Cell proliferation was measured using WST-1 reagent from Abcam (#ab65473). At Day 0, cells were plated in 96-well tissue culture plate. At day 1, the cells were starved in serum (100 μL/well). At Day 2 cells were treated with different drugs or DMSO at the indicated concentrations, in quadruplicates. 48 h post treatment, WST-1 reagent (10 μL/well) was added. The plate was read at TO at 450 nm on a Multiskan FC Counter (ThermoScientific), then every hour until O.D reach 1.0. Cell proliferation is expressed as percent of the absorbance after subtraction to background (T0).

Cells were treated for 48 h before the WST-1 assay with variable concentration of HA15 and different concentration of the indicated anticancer agent.

To evaluate the effect of HA15 in combination with another anticancer agent, we compared observed and expected responses obtained from the combination treatment. Bliss model was used to predict the combined effect of each drug.

The expected effect (Eexp) of the combination was estimated from each separate drug effect.

The results are provided in the Tables below. “SE” corresponds to a significant efficacy of the combination of HA15 with the identified anticancer agent.

Doxorubicin

| | HA15 | HA15 |
|---|---|---|
| Cell lines | ⅗ μM | 10 μM |
| AGS (gastric) | SE | SE |
| SH10TC (gastric) | SE | SE |

Docetaxel

| | HA15 | HA15 |
|---|---|---|
| Cell lines | ⅗ μM | 10 μM |
| SH10TC (gastric) | SE | SE |

5-FU

| | HA15 | HA15 |
|---|---|---|
| Cell lines | ⅗ μM | 10 μM |
| AGS (gastric) | SE | SE |
| SH10TC (gastric) | SE | SE |
| NCI-N87 (gastric) | SE | SE |
| Panc-1 (pancreas) | SE | SE |
| HT29 (colorectal) | SE | SE |

| | Gemcitabine |
|---|---|
| Cell lines | HA15 10 μM |
| Panc-1 (pancreas) | SE |
| MiaPaCa-2 | SE |
| (pancreas) | |

Cisplatin

| | HA15 | HA15 |
|---|---|---|
| Cell lines | ⅗ μM | 10 μM |
| AGS (gastric) | SE | SE |
| SH10TC (gastric) | SE | SE |

Oxaliplatin

| | HA15 | HA15 |
|---|---|---|
| Cell lines | ⅗ μM | 10 μM |
| NCI-N87 (gastric) | SE | SE |
| HT29 (colorectal) | SE | SE |

Oxaliplatin + 5FU

| | HA15 | HA15 |
|---|---|---|
| Cell lines | ⅗ μM | 10 μM |
| NCI-N87 (gastric) | SE | SE |
| HT29 (colorectal) | SE | SE |

Irinotecan metabolite (SN-38)

| | HA15 | HA15 |
|---|---|---|
| Cell lines | ⅗ μM | 10 μM |
| AGS (gastric) | SE | SE |
| Pan-02 | SE | SE |
| (pancreas) | | |

Bortezomib

| | HA15 | HA15 |
|---|---|---|
| Cell lines | ⅗ μM | 10 μM |
| KMM1 | SE | SE |
| (myeloma) | | |
| U266 (myeloma) | SE | SE |
| 8226 (myeloma) | SE | SE |

Vemurafenib

| | HA15 | HA15 |
|---|---|---|
| Cell lines | ⅗ μM | 10 μM |
| A375 (melanoma) | SE | SE |
| Mel624 | SE | SE |
| HT29 (colorectal) | SE | SE |

Cobimetinib +/− Vemurafenib

| | HA15 | HA15 |
|---|---|---|
| Cell lines | ⅗ μM | 10 μM |
| Mel624 | SE | SE |
| (melanoma) | | |
| HT29 (colorectal) | SE | SE |

These results show that HA15 allows potentiating the response to all several anticancer treatments including chemotherapies and targeted therapies. It can therefore be concluded that HA15 allows potentiating the effects of anticancer treatments.

1. A method of treating cancer in a patient who has been identified as overexpressing GRP78 said method comprising administering a combination of (i) an anticancer treatment with (ii) compound of formula (I): 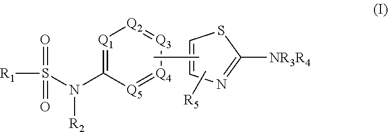
wherein
Q₁ to Q₅ identical or different represent CR₆
R₁ represents C₆-C₁₀ aryl comprising one or two fused rings, wherein from 2 to 5 carbon atoms may be replaced with a heteroatom selected from O, S, N and NR₆, and eventually substituted with from 5 to 11 substituents selected from R₆, halo, CN, NO₂, CF₃, OCF₃, COOR₆, OCOR₆, SO₂NR₆R₇, CONR₆R₇, NR₆R₇, NR₆COR₇, (CH₂)ₚ, NR₆R₇, (CH₂)ₚ₋ OR₆ and (CH₂)_{P}SR₆,
R₂ is SO₂R₁ or R₆
R₃ and R₄ identical or different are selected from CORs and R₆
R₅ represents R₆, aryl, OR₆, SR₆, halo, CN, NO₂, CF₃, OCF₃, COOR₆, SO₂NR₆R₇, CONR₆R₇, NR₆R₇ and NHCOR₆,
R₆ and R₇ identical or different represent H or alkyl
R₈ is selected from H, alkyl, cycloalkyl, aryl, alkylaryl, wherein aryl may be substituted with from one to four R₅ substituents identical or different,
or R₈ represents —(CH₂)_{q}—NR₆R₇,
p represents an integer from 0 to 6,
q represents an integer from 0 to 6,
wherein the thiazolyl group is linked to the 6-member group in meta or para position with respect to the sulfonamide group and wherein the thiazolyl group is linked to the 6-member group in position α or β with respect to the S atom,
for use in the treatment of cancer in a patient who has been identified as overexpressing GRP78.

2. The method according to claim 1, wherein said anticancer treatment is selected from chemotherapeutic agents, targeted therapies and immunotherapeutic agents such as an immune checkpoint inhibitor.

3. The method according to claim 1, wherein said anticancer treatment is an immune checkpoint inhibitor which is an anti-PD-1 or an anti-PDL1 antibody.

4. The method according to claim 1, wherein said compound of formula (I) is selected from the group consisting of:
N-(4-(3-(5-(dimethylamino)naphthalene-1-sulfonamido)phenyl)thiazol-2-yl)acetamide 5-(dimethylamino)-N-(3-(2-(methylamino)thiazol-4-yl)phenyl)naphthalene- 1-sulfonamide
N-(4-(3-(5-(dimethylamino)naphthalene-1-sulfonamido)phenyl)thiazol-2-yl)-4-methylbenzamide
N-(3-(2-aminothiazol-4-yl)phenyl)-5-(dimethylamino)naphthalene- 1-sulfonamide
N-(4-(3-(5-(dimethylamino)naphthalene-1-sulfonamido)phenyl)thiazol-2-yl)benzamide
N-(4-(3-(5-(dimethylamino)naphthalene-1-sulfonamido)phenyl)thiazol-2-yl)pivalamide
2-fluoro-N-(3-(2-(methylamino)thiazol-4-yl) phenyl)benzenesulfonamide
N-(4-(4-(naphthalene-2-sulfonamido)phenyl)thiazol-2-yl)acetamide
N-(4-(4-(5-(dimethylamino)naphthalene-1-sulfonamido)phenyl)thiazol-2-yl)acetamide
N-(4-(4-(2-fluorophenylsulfonamido)phenyl)thiazol-2-yl)acetamide
N-(4-(4-(2,4-difluorophenylsulfonamido)phenyl)thiazol-2-yl)acetamide
N-(4-(4-(3-(trifluoromethyl)phenylsulfonamido)phenyl)thiazol-2-yl)acetamide
N-(4-(3-(3-(trifluoromethyl)phenylsulfonamido)phenyl)thiazol-2-yl)acetamide
N-(4-(4-(4-(trifluoromethyl)phenylsulfonamido)phenyl)thiazol-2-yl)acetamide
N-(4-(3-(4-methylphenylsulfonamido) phenyl)thiazol-2-yl)acetamide
N-(4-(3-(2-nitrophenylsulfonamido) phenyl)thiazol-2-yl)acetamide
N-(4-(3-(3-nitrophenylsulfonamido) phenyl)thiazol-2-yl)acetamide
N-(4-(3-(phenylsulfonamido)phenyl)thiazol-2-yl)acetamide
N-(4-(3-(methylsulfonamido)phenyl)thiazol-2-yl)acetamide
N-(4-(4-(4-methylphenylsulfonamido) phenyl)thiazol-2-yl)acetamide
and
N-(4-(3-(5-(dimethylamino)naphthalene-1-sulfonamido)phenyl)thiazol-2-yl)-6-amino- hexanamide.

5. The method according to claim 1, wherein said compound is of formula (III):

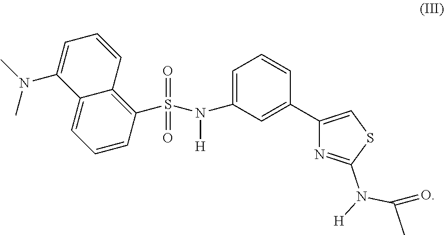 [SMILES: CC(=O)Nc1nc(-c2cccc(NS(=O)(=O)c3cccc4c(N(C)C)cccc34)c2)cs1]

6. The method according to claim 1, wherein said cancer is selected from the group consisting of breast cancer, bladder cancer, cervical cancer, colorectal cancer, pancreatic cancer head and neck cancer, Hodgkin lymphoma, liver cancer, lung cancer, kidney cancer, skin cancer, and stomach cancer.

7. The method according to claim 1, wherein said cancer is colorectal cancer.

8. The method according to claim 1, wherein said cancer is skin cancer.

9. The method according to claim 1, wherein said cancer is a non-immunogenic tumor.

10. The method according to claim 1, wherein said cancer is resistant to immunotherapy.

11. The method according to claim 10, wherein said cancer is resistant to immune checkpoint inhibitors.

12. A kit of parts comprising:
an anticancer agent; and
a compound of formula (I) 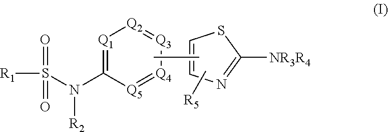
wherein
Q1 to Q5 identical or different represent CR6
R1 represents C6-C10 aryl comprising one or two fused rings, wherein from 2 to 5 carbon atoms may be replaced with a heteroatom selected from O, S, N and NR6, and eventually substituted with from 5 to 11 substituents selected from R₆, halo, CN, NO2, CF3, OCF3, COOR6, OCOR6, SO2NR6R7, CONR6R7, NR6R7, NR6COR7, (CH2)p- NR6R7, (CH2)p- OR6 and (CH2)PSR6,
R2 is SO2R1 or R6
R3 and R4 identical or different are selected from COR8 and R6
R5 represents R6, aryl, OR6, SR6, halo, CN, NO2, CF3, OCF3, COOR6, SO2NR6R7, CONR6R7, NR6R7 and NHCOR6,
R6 and R7 identical or different represent H or alkyl
R8 is selected from H, alkyl, cycloalkyl, aryl, alkylaryl, wherein aryl may be substituted with from one to four R5 substituents identical or different,
or R₈ represents —(CH2)q-NR6R7,
p represents an integer from 0 to 6,
q represents an integer from 0 to 6,
wherein the thiazolyl group is linked to the 6-member group in meta or para position with respect to the sulfonamide group and wherein the thiazolyl group is linked to the 6-member group in position α or β with respect to the S atom.

13. A method of treating cancer in a patient who has been identified as overexpressing GRP78 comprising using a kit of parts of claim 12.

14. The method of claim 8 wherein the skin cancer is melanoma.
